SEQUENCE LISTING (43 sequences)

SEQ ID NO: 1 - DNA (cDNA), 40 bp; both-stranded, both
caggatccacatggtgcttaacggcgacatccacactaag

SEQ ID NO: 2 - DNA (cDNA), 27 bp; both-stranded, both
gttaacttcttgtgcggtctcaatgac

SEQ ID NO: 3 - protein, 11 aa
AKAITFGILYG

SEQ ID NO: 4 - protein, 11 aa
AKTFIYGFLYG

SEQ ID NO: 5 - protein, 11 aa
AKTINFGVLYG

SEQ ID NO: 6 - protein, 11 aa
AKAINFGLIYG

SEQ ID NO: 7 - protein, 4 aa
TFIY

SEQ ID NO: 8 - protein, 4 aa
AITF

SEQ ID NO: 9 - protein, 4 aa
TINF

SEQ ID NO: 10 - DNA (cDNA), 45 bp; both-stranded, both
tcaggctgctaaaacattcatctacggtatactgtatggttctgg

SEQ ID NO: 11 - DNA (cDNA), 33 bp; both-stranded, both
gtagaggaccccgtaattaatggtcttggccgc

SEQ ID NO: 12 - protein, 11 aa
GKMVNFSIIYG

SEQ ID NO: 13 - protein, 4 aa
MVNF

SEQ ID NO: 14 - DNA (genomic), 694 bp; double-stranded, both
  CDS 2..694
ggatccagactggtggatcgtcagtgcggattattcccaaatagaactcagaatcctcgctcatctcagtggtgatgagaaccttgtgaaggccttcgaggagggcatcgatgtgcacaccttgactgcctccaggatctacaacgtaaagccagaagaagtgaacgaagaaatgcgacgggttggaaagatggtgaacttctctataatatacggtgtcacaccgtacggtctttctgtgagacttggaataccggttaaagaagcagaaaagatgattatcagctatttcacactgtatccaaaggtgcgaagctacatccagcaggttgttgcagaggcaaaagagaagggctacgtcaggactctctttggaagaaaaagagatattccccagctcatggcaagggacaagaacacccagtccgaaggcgaaagaatcgcaataaacacccccattcagggaactgcggcagatataataaaattggctatgatagatatagacgaggagctgagaaaaagaaacatgaaatccagaatgatcattcaggttcatgacgaactggtcttcgaggttcccgatgaggaaaaagaagaactagttgatctggtgaagaacaaaatgacaaatgtggtgaaactctctgtgcctcttgaggttgacataagcatcggaaaaagctggtcttga

SEQ ID NO: 15 - protein, 230 aa
DPDWWIVSADYSQIELRILAHLSGDENLVKAFEEGIDVHTLTASRIYNVKPEEVNEEMRRVGKMVNFSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 16 - DNA (cDNA), 31 bp; both-stranded, both
gtatattatagagtagttaaccatctttcca

SEQ ID NO: 17 - DNA (cDNA), 26 bp; both-stranded, both
gacgtttcaagcgctagggcaaaaga

SEQ ID NO: 18 - protein, 11 aa
GKMVNFSIIYG

SEQ ID NO: 19 - protein, 24 aa
GSRLVDRQCGLFPNRTQNPRSSQW

SEQ ID NO: 20 - protein, 60 aa
EPCEGLRGGHRCAHLDCLQDLQRKARRSERRNATGWKDGELLYNIRCHTVRSFCETWNTG

SEQ ID NO: 21 - protein, 99 aa
RSRKDDYQLFHTVSKGAKLHPAGCCRGKREGLRQDSLWKKKRYSPAHGKGQEHPVRRRKNRNKHPHSGNCGRYNKIGYDRYRRGAEKKKHEIQNDHSGS

SEQ ID NO: 22 - protein, 8 aa
RTGLRGSR

SEQ ID NO: 23 - protein, 6 aa
GKRRTS

SEQ ID NO: 24 - protein, 16 aa
SGEEQNDKCGETLCAS

SEQ ID NO: 25 - protein, 9 aa
HKHRKKLVL

SEQ ID NO: 26 - protein, 13 aa
IQTGGSSVRIIPK

SEQ ID NO: 27 - protein, 14 aa
NSESSLISVVMRTL

SEQ ID NO: 28 - protein, 11 aa
RPSRRASMCTP

SEQ ID NO: 29 - protein, 7 aa
LPPGSTT

SEQ ID NO: 30 - protein, 4 aa
SQKK

SEQ ID NO: 31 - protein, 10 aa
TKKCDGLERW

SEQ ID NO: 32 - protein, 10 aa
YTVSHRTVFL

SEQ ID NO: 33 - protein, 11 aa
DLEYRLKKQKR

SEQ ID NO: 34 - protein, 56 aa
LSAISHGIQRCEATSSRLLQRQKRRATSGLSLEEKEIFPSSWQGTRTPSPKAKESQ

SEQ ID NO: 35 - protein, 10 aa
TPPFRELRQI

SEQ ID NO: 36 - protein, 4 aa
EKET

SEQ ID NO: 37 - protein, 19 aa
SFRFMTNWSSRFPMRKKKN

SEQ ID NO: 38 - protein, 9 aa
NSLCLLRLT

SEQ ID NO: 39 - protein, 7 aa
ASEKAGL

SEQ ID NO: 40 - protein, 11 aa
PSFALDLETSS

SEQ ID NO: 41 - protein, 11 aa
PYFAFDTETDS

SEQ ID NO: 42 - protein, 11 aa
GPVAFDSETSA

SEQ ID NO: 43 - protein, 10 aa
MIVSDIEANA

(19) United States
(12) Patent Application Publication
(10) Pub. No.: US 2005/0112637 A1
(43) Pub. Date: May 26, 2005

(54) Mutant DNA polymerases and uses thereof

(76) Inventor: Deb K. Chatterjee, North Potomac, MD (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/947,352

(22) Filed: Sep. 23, 2004

Related U.S. Application Data
(62) Division of application No. 09/558,421, filed on Apr. 26, 2000.
Application No. 09/558,421 is a continuation of application No. 08/576,759, filed on Dec. 21, 1995, now abandoned.
Application No. 08/576,759 is a continuation of application No. 08/537,397, filed on Oct. 2, 1995, now abandoned.
Application No. 08/537,397 is a continuation-in-part of application No. 08/525,057, filed on Sep. 8, 1995, now abandoned.

(51) Int. Cl. C12Q 1/68; C07H 21/04; C12N 9/22; C12N 1/21; C12N 15/74

(52) U.S. Cl. 435/6; 435/69.1; 435/199; 435/252.33; 435/320.1; 536/23.2

(57) ABSTRACT

The present invention relates to mutant DNA polymerases which incorporate dideoxynucleotides with about the same efficiency as deoxynucleotides. The present invention also related to mutant DNA polymerases which also have substantially reduced 5′-to-3′ exonuclease activity or 3′-to-5′ exonuclease activity. The invention also relates to DNA molecules coding for the mutant DNA polymerases, and hosts containing the DNA molecules.

### CROSS REFERENCE TO RELATED APPLICATIONS

This application is a continuation of Ser. No. 08/537,397, filed Oct. 2, 1995, entitled Mutant DNA Polymerases and Uses Thereof, which is a continuation-in-part of Ser. No. 08/525,057 of Deb K. Chattejee, filed Sep. 8, 1995, also entitled Mutant DNA Polymerases and the Use Thereof. The content of both of these applications is specifically incorporated herein by reference.

### FIELD OF THE INVENTION

This invention relates to molecular cloning and expression of mutant DNA polymerases that are particularly useful in DNA sequencing reactions.

### BACKGROUND OF THE INVENTION

DNA polymerases synthesize the formation of DNA molecules from deoxynucleotide triphosphates using a complementary template DNA strand and a primer. DNA polymerases synthesize DNA in the 5′-to-3′ direction by successively adding nucleotides to the free 3′-hydroxyl group of the growing strand. The template strand determines the order of addition of nucleotides via Watson-Crick base pairing. In cells, DNA polymerases are involved in repair synthesis and DNA replication.

Bacteriophage T5 induces the synthesis of its own DNA polymerase upon infection of its host, Escherichia coli. The T5 DNA polymerase (T5-DNAP) was purified to homogeneity by Fujimura R K & Roop B C, J Biol. Chem. 25:2168-2175 (1976). T5-DNAP is a single polypeptide with a molecular weight of about 96 kilodaltons. This polymerase is highly processive and, unlike T7 DNA polymerase, does not require thioredoxin for its processivity (Das S K & Fujimura R K, J. Biol. Chem. 252:8700-8707 (1977); Das S K & Fujimura R K, J. Biol. Chem. 254:1227-1237 (1979)).

Fujimura R K et al., J. Virol. 53:495-500 (1985) disclosed the approximate location of the T5-DNAP gene on the physical restriction enzyme map generated by Rhoades, J. Virol. 43:566-573 (1982). DNA sequencing of the fragments of this corresponding region was disclosed by Leavitt & Ito, Proc. Natl. Acad Sci. USA 86:4465-4469 (1989). However, the authors did not reassemble the sequenced fragments to obtain expression of the polymerase.

Copending application Ser. No. 08/370,190, filed Jan. 9, 1995, discloses a DNA polymerase from an eubacterium, Thermotoga neapolitana (Tne). A partial restriction map and a partial DNA sequence of this DNA polymerase gene have been established.

An oligonucleotide-directed, site-specific mutation of a T7 DNA polymerase gene was disclosed by Tabor S & Richardson CC, J. Biol. Chem. 264:6447-6458 (1989).

The existence of a conserved 3′-to-5′ exonuclease active site present in a number of DNA polymerases is discussed in Bernard A et al., Cell 59:219-228 (1989). T5 DNA polymerase which lacks 3′-to-5′ exonuclease activity is disclosed in U.S. Pat. No. 5,270,179.

In molecular biology, DNA polymerases have several uses. In cloning and gene expression experiments, DNA polymerases are used to synthesize the second strand of a single-stranded circular DNA annealed to an oligonucleotide primer containing a mutated nucleotide sequence. DNA polymerases have also been used for DNA sequencing by the Sanger Dideoxy method. For example, the Klenow fragment, Taq DNA polymerase and T7 DNA polymerase lacking substantial exonuclease activity, are useful for DNA sequencing. Such DNA sequencing procedures are carried out by annealing a primer to a DNA molecule to be sequenced, incubating the annealed mixture with a DNA polymerase, and four deoxynucleotide triphosphates in four vessels each of which contains a different DNA synthesis terminating agent (e.g. a dideoxynucleoside triphosphate). The agent terminates at a different specific nucleotide base in each of the four vessels. The DNA products of the incubating reaction are separated according to their size so that at least part of the nucleotide base sequence of the DNA molecule can be determined.

Residues in DNA polymerases important for binding of nucleotides have been investigated by Polesky, A. H. et al., J. Biol. Chem. 265:14579-14591 (1990) and Astatke M et al., J. Biol. Chem. 270:1945-1954 (1995).

While several DNA polymerases are known, there exists a need in the art for additional DNA polymerases having properties suitable for DNA synthesis, DNA sequencing, and DNA amplification.

### SUMMARY OF THE INVENTION

The present invention helps satisfy these needs in the art of providing additional DNA polymerases and uses therefor. This invention is related to the discovery that it is possible to prepare mutant DNA polymerases that incorporate dideoxynucleotides into a synthesized DNA molecule with about the same efficiency that deoxynucleotides are incorporated. Such mutant DNA polymerases may be used to prepare sequencing ladders having bands of approximately equal intensity.

Thus, the present invention is related to a mutant DNA polymerase that incorporates dideoxynucleotides with about the same efficiency as deoxynucleotides, wherein the native DNA polymerase favors the incorporation of deoxynucleotides over dideoxynucleoties. Examples of the mutant DNA polymerase include a mutant Klenow fragment of DNA polymerase, e.g. of E. coli, a mutant T5 DNA polymerase, a mutant Taq polymerase, a mutant Thermatoga maritima (Tma) DNA polymerase (U.S. Pat. No. 5,374,553), and a mutant of Tne polymerase.

The invention also relates to a DNA molecule which codes for the mutant DNA polymerase of the present invention as well as host cells comprising the DNA molecule.

The invention also relates to a method for producing a protein, wherein said protein has a mutant DNA polymerase activity and incorporates dideoxynucleotides with about the same efficiency as deoxynucleotides, said method comprising the steps of:
(i) culturing a host cell containing the DNA molecule of the invention, and
(ii) isolating said protein from said host cell.

Examples of such mutant DNA polymerase proteins include mutant T5 DNA polymerase, wherein Tyr⁵⁷⁰ is substituted for Phe⁵⁷⁰ of native T5 DNA polymerase; mutant Taq DNA polymerase, wherein Tyr⁶⁶⁷ is substituted for Phe⁶⁶⁷ of native Taq DNA polymerase; mutant Klenow fragment DNA polymerase, wherein Tyr⁷⁶² is substituted for Phe⁷⁶² of Klenow DNA polymerase; mutant Tne DNA polymerase, wherein Tyr⁶⁷ is substituted for Phe⁶⁷ of Tne DNA polymerase, as numbered in FIG. 4; and a mutant Tma DNA polymerase, wherein Tyr⁷³⁰ is substituted for Phe⁷³⁰.

In addition, this invention also relates to mutant DNA polymerases, that, in addition to incorporating dideoxynucleotides into a DNA molecule about as efficiently as deoxynucleotides, has substantially reduced 5′-to-3′ exonuclease activity, substantially reduced 3′-to-5′ exonuclease activity, or both substantially reduced 5′-to-3′-exonuclease activity and substantially reduced 3′-to-5′ exonuclease activity. By way of example, such a mutant DNA polymerase can be a T5 DNA polymerase, a Tne DNA polymerase, a Klenow fragment DNA polymerase, a Taq DNA polymerase or a Tma DNA polymerase. This invention also relates to DNA molecules coding for mutant DNA polymerases with substantially reduced exonuclease activity, host cells comprising the DNA molecule, and methods of producing these mutant DNA polymerases.

### BRIEF DESCRIPTION OF THE DRAWINGS

FIG. 1 is a map of the T5 DNA polymerase expression vector pSportT5#3.

FIG. 2 is a map of the Taq DNA polymerase expression vector pTTQ-Taq.

FIG. 3 is a restriction map of plasmids pSport-Tne and pUC-Tne. The locations of the Tne DNA polymerase, as well as the region containing the O-helix homologous sequence, are indicated.

FIG. 4 depicts the nucleotide and deduced amino acid sequences, in all 3 reading frames, of the C-terminal portion, including the O helix region, of the Tne DNA polymerase gene.

FIG. 5A schematically depicts the construction of plasmids pUC-Tne (3′-5′) and pUC-TneFY from pUC-Tne.

FIG. 5B schematically depicts the construction of plasmids pTrcTne35 and pTrcTneFY from pUCTne(3′-5′) and pUC-TneFY, respectively.

FIG. 6 schematically depicts the construction of pTrcTne35FY from pUC-Tne (3′-5′) and pUC-TneFY.

FIG. 7 schematically depicts the construction of plasmids pTTQTne535FY and pTTQTne5FY.

### DETAILED DESCRIPTION OF THE INVENTION

One of the applications of DNA polymerases, particularly the E. coli DNA polymerase I family, is in DNA sequencing. Of the known polymerases, the large fragment (Klenow fragment) of E. coli DNA polymerase I, T7 DNA polymerase, and Taq DNA polymerase are used more frequently than other DNA polymerases.

The DNA polymerase of E. coli bacteriophage T5 has recently been cloned and expressed. See U.S. Pat. Nos. 5,270,179 and 5,047,342. The T5 DNA polymerase is a highly processive polymerase and does not require any accessory protein, such as thoiredoxin, to be processive. Although T5 DNA polymerase is capable sequencing DNA in the presence of dideoxynucleoside triphosphates, it requires 20-30 fold more concentrated solutions compared to the concentration for the deoxynucleotide triphosphates to generate sequencing ladders. DNA sequencing with other polymerases such as Klenow fragment and Taq DNA polymerase also requires more dideoxynucleotides, similar to T5 DNA polymerase, to generate sequencing ladders.

T7 DNA polymerase, on the other hand, requires thioredoxin for processivity and almost eqimolar or less concentrations of dideoxynucleotides to deoxynucleotides to generate suitable sequencing ladders. The most important difference in the sequencing ladder produced by T7 DNA polymerase compared to others is that it produces bands with equal intensity throughout the sequence, while Klenow fragment, T5 DNA polymerase, Tne DNA polymerase and Taq DNA polymerases produced sequence dependent uneven band intensity. Thus, T7 DNA polymerase is more non-discriminating and more efficiently incorporates dideoxynucleotides into DNA; while T5, Taq, Tne, and Tma DNA polymerase, and Klenow fragment are more discriminating and incorporate dideoxynucleotides inefficiently.

The Tne DNA polymerase has a molecular weight of about 100 kDa. This polymerase is extremely thermostable, showing more than 50 percent activity after being heated for 60 minutes at 90° C. with or without detergent. Thus, the Tne DNA polymerase is more thermostable than Taq polymerase.

The Tne DNA polymerase of the invention can be isolated from any strain of Thermatoga neapolitana, which produces a DNA polymerase having a molecular weight of about 100 kDa. The most preferred Thermatoga strain for isolating the DNA polymerase of the invention was isolated from an African continental solfataric spring (Winberger et al., Arch. Microbiol. 151:506-512 (1989)) and may be obtained from the Deutsche Sammalung von Microorganismen und Zellkulturan GmbH, Braunschweig, Fed. Rep. Germany, as Deposit No. 5068.

The recombinant clone containing the gene encoding DNA polymerase (DH10B/pUC-Tne) was deposited on Sep. 30, 1994, with the Patent Culture Collection, Northern Regional Research Center, USDA, 1815 N. University Street, Peoria, Ill. 61604, USA, as Deposit No. NRRL B-21338.

The amino acid sequence comparison of all of these DNA polymerases suggests that all contain the conserved dNTP binding amino acids. Crystal structure as well as biochemical studies suggest that several amino acids, such as Lys and Tyr, present in the O-helix are important in dNTP binding. Both of these amino acids and several other amino acids are conserved in Klenow fragment, T5, Taq, Tne and T7 DNA polymerases (Poleskey, A. H. et. al., J. Biol. Chem. 265:14579-14591 (1990)). Thus, amino acid(s) directly or indirectly involved in dNTP binding may be responsible for discrimination of dideoxynucleotides. By incorporating active regions of T7 DNA polymerase (which do not discriminate) into other polymerases, mutant DNA polymerases were constructed, which do not discriminate against dideoxynucleotides. The invention relates to this discovery.

Amino acid residues of T5 DNA polymerase are numbered herein as numbered in U.S. Pat. No. 5,270,179 and Leavitt and Ito, Proc. Natl. Acad Sci USA 86.:4465-4469 (1989).

Amino acid residues of T7 DNA polymerase are numbered as numbered by Dunn and Studier, J. Mol. Biol. 166:477-535 (1983).

Amino acid residues of Taq DNA polymerase are as numbered in U.S. Pat. No. 5,079,352.

Amino acid residues of the Klenow fragment of E. coli are as numbered by Joyce, C. M. et al., J. Biol. Chem. 257:1958-1964 (1982).

Amino acid residues of Thermatoga neapolitana (Tne) are numbered as in U.S. Ser. No. 08/370,170, filed Jan. 9, 1995, which is specifically incorporated herein by reference.

Amino acid residues of Thermatoga maritima (Tma) DNA polymerase are numbered as in U.S. Pat. No. 5,374,553.

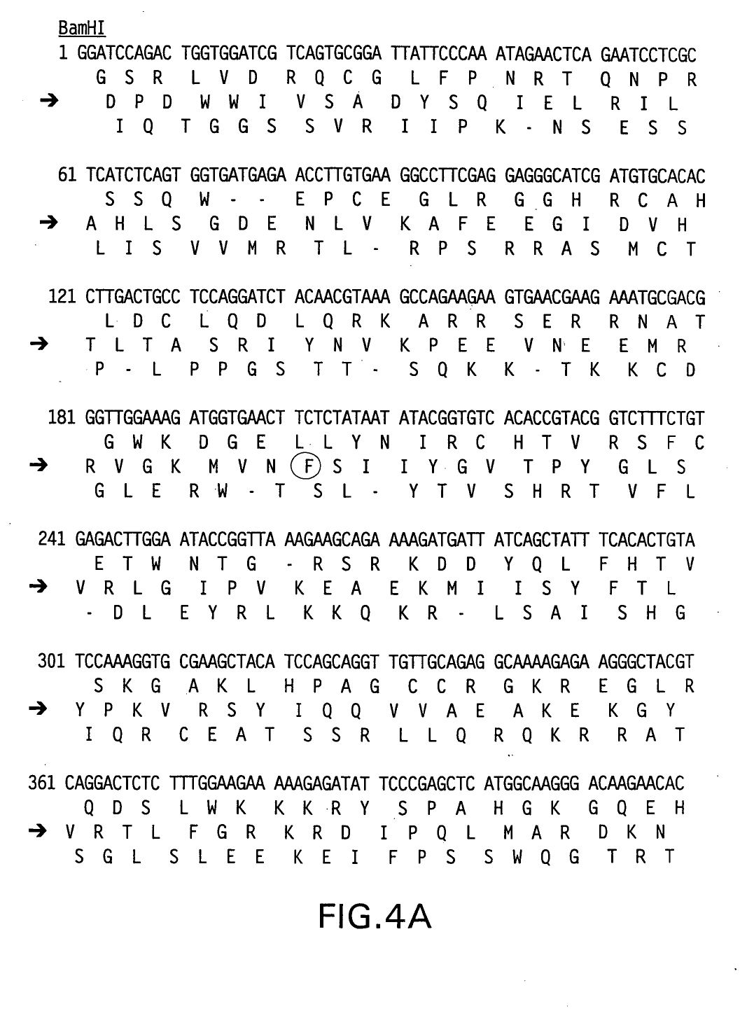

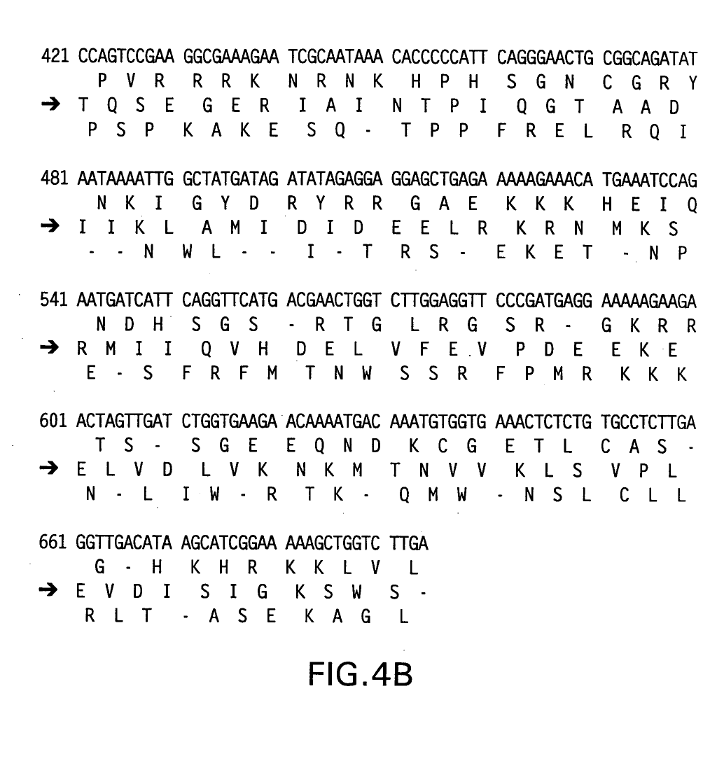

In addition to the DNA polymerases mentioned above, it is also possible to prepare the following mutant DNA polymerases:

| Enzyme or source | Mutation position |
|---|---|
| E. coli DNA polymerase I | 762 |
| Streptococcus pneumoniae | 711 |
| Thermus aquaticus | 667 |
| Thermus flavus | 666 |
| Thermus thermophilus | 669 |
| Deinococcus radiodurans | 747 |
| Bacillus caldotenax | 711 |
| E. coli bacteriophage T5 | 570 |
| mycobacteriophage L5 | 438 |
| E. coli bacteriophage SP01 | 692 |
| E. coli bacteriophage SP02 | 447 |
| Thermatoga neapolitana | 67 |
| | [FIG. 4] |
| Thermatoga maritima | 730 |

The change in amino acid at the mutation positions above is from phenylalanine to tyrosine except for bacteriophage SP02, where the change is from leucine to tyrosine. Coordinates are as used by Polesky, A. H. et al., J. Biol. Chem. 265:14579-14591 (1990) and Astatke M et al., J. Biol. Chem. 270:1945-1954 (1995).

The following terms are defined in order to provide a clear and consistent understanding of their use in the specification and the claims. Other terms are well known to the art so that they need not be defined herein.

“Structural gene” is a DNA sequence that is transcribed into messenger RNA and is then translated into a sequence of amino acid residues characteristic of a specific polypeptide.

“Soluble” refers to the physical state of a protein upon expression in a host cell, i.e., the protein has the ability to form a solution in vivo. As used herein, a protein is “soluble” if the majority (greater than 50%) of the protein produced in the cell is in solution and is not in the form of insoluble inclusion bodies.

“Nucleotide” is a monomeric unit of DNA or RNA consisting of a sugar moiety, a phosphate, and a nitrogenous heterocyclic base. The base is lined to the sugar moiety via the glycosidic carbon (1′ carbon of the pentose). The combination of a base and a sugar is called a nucleoside. Each nucleotide is characterized by its base. The four DNA bases are adenine (A), guanine (G), cytosine (C), and thymine (T). The four RNA bases are A, G, C and uracil (U).

“Processive” is a term of art referring to an enzyme's property of acting to synthesize or hydrolyze a polymer without dissociating from the particular polymer molecule. A processive DNA polymerase molecule can add hundreds of nucleotides to a specific nucleic acid molecule before it may dissociate and start to extend another DNA molecule. Conversely, a non-processive polymerase will add as little as a single nucleotide to a primer before dissociating from it and binding to another molecule to be extended. For the purposes of the present invention, processive refers to enzymes that add, on the average, at least 100, and preferably, about 200 or more, nucleotides before dissociation.

“Thioredoxin” is an enzyme well known to the art that is involved in oxidation and reduction reactions. It is also required as a subunit for T7 DNA polymerase activity. “Thioredoxin-independent” refers to the ability of and polymerase to be processive in the absence of thioredoxin.

“Promoter” is a term of art referring to sequences necessary for transcription. It does not include ribosome binding sites and other sequences primarily involved in translation.

“Gene” is a DNA sequence that contains information necessary to express a polypeptide or protein. A gene may include homologous or heterologous control elements such as promoters, enhancers, and ribosome binding sites.

“Heterologous” refers herein to two molecules having different origins; i.e. not, in nature, being genetically or physically linked to each other. “Heterologous” also describes molecules that while physically or genetically linked together in nature, are linked together in a substantially different way than is found in nature.

“Homology”, as used herein, refers to the comparison of two different nucleic acid sequences. For the present purposes, assessment of homology is as a percentage of identical bases, not including gaps introduced into the sequence to achieve good alignment. Per cent homology may be estimated by nucleic acid hybridization techniques, as is well understood in the art as well as by determining and comparing the exact base order of the two sequences.

“Mutation” is any change that alters the DNA or amino acid sequence. As used herein, a mutated sequence may have single or multiple changes that alter the nucleotide sequence of the DNA or the amino acid sequence of the protein. Alterations of the DNA or amino acid sequence include deletions (loss of one or more nucleotides or amino acids in the sequence), substitutions (substituting a different nucleotide or amino acid for the original nucleotide or amino acid along the sequence) and additions (addition of new nucleotides or amino acids in the original sequence).

“Purifying” refers herein to increasing the specific activity of an enzyme over the level produced in a culture in terms of units of activity per weight of protein. This term does not imply that a protein is purified to homogeneity. Purification schemes for DNA polymerases are known to the art.

“Expression” is the process by which a polypeptide is produced from a structural gene. It includes transcription of the gene into messenger RNA (mRNA) and the translation of such mRNA into polypeptide(s).

“Substantially pure” means that the desired purified molecule, e.g., enzyme or polypeptide, is essentially free from contaminating cellular components which are associated with the desired enzyme or polypeptide in nature. Contaminating cellular components may include, but are not limited to, phosphatases, exonucleases, endonucleases or other amino acid sequences normally associated with the desired enzyme or polypeptide.

“Origin of replication” refers to a DNA sequence from which DNA replication is begun, thereby allowing the DNA molecules which contain said origin to be maintained in a host, i.e., replicate autonomously in a host cell.

“Host” is any prokaryotic or eukaryotic microorganism that is the recipient of a DNA molecule. The DNA molecule may contain, but is not limited to, a structural gene, expression control elements, e.g. a promoter and/or an origin of replication.

“3′-to-5′ exonuclease activity” is an enzymatic activity well known to the art. This activity is often associated with DNA polymerases, and is thought to be involved in a DNA replication “editing” or correction mechanism.

“5′ to 3′ exonuclease activity” is also an enzymatic activity well known in the art. This activity is often associated with DNA polymerases, such as E. coli PolI and PolIII.

A “DNA polymerase substantially reduced in 3′-to-5′ exonuclease activity” is defined herein as either (1) a mutated DNA polymerase that has about or less than 10%, or preferably about or less than 1%, of the 3′-to-5′ exonuclease activity of the corresponding unmutated, wild-type enzyme, or (2) a DNA polymerase having a 3′-to-5′ exonuclease specific activity which is less than about 1 unit/mg protein, or preferably about or less than 0.1 units/mg protein. A unit of activity of 3′-to-5′ exonuclease is defined as the amount of activity that solubilizes 10 nmoles of substrate ends in 60 min. at 37° C., assayed as described in the “BRL 1989 Catalogue & Reference Guide”, page 5, with HhaI fragments of lambda DNA 3′-end labeled with ³[H]dTTP by terminal deoxynucleotidyl transferase (TdT). Protein is measured by the method of Bradford, Anal. Biochem. 72:248 (1976). As a means of comparison, natural, wild-type T5-DNAP or T5-DNAP encoded by pTTQ19-T5-2 has a specific activity of about 10 units/mg protein while the DNA polymerase encoded by pTTQ19-T5-2Exo⁻) (U.S. Pat. No. 5,270,179) has a specific activity of about 0.0001 units/mg:protein, or 0.001% of the specific activity of the unmodified enzyme, a 10⁵-fold reduction.

A “DNA polymerase substantially reduced in 5′-to-3′ exonuclease activity” is defined herein as either (1) a mutated DNA polymerase that has about or less than 10%, or preferably about or less than 1%, of the 5′-to-3′ exonuclease activity of the corresponding unmutated, wild-type enzyme, or (2) a DNA polymerase having 5′-to-3′ exonuclease specific activity which is less than about 1 unit mg protein, or preferably about or less than 0.1 units/mg protein.

Both of these activities, 3′-to-5′ exonuclease activity and 5′-to-3′ exonuclease activity, can be observed on sequencing gels. Active 5′-to-3′ exonuclease activity will produce nonspecific ladders in a sequencing gel by removing nucleotides from growing primers. 3′-to-5′ exonuclease activity can be measured by following the degradation of radiolabeled primers in a sequencing gel. Thus, the relative amounts of these activities, e.g. by comparing wild-type and mutant polymerases, can be determined from these charateristics of the sequencing gel.

As used herein, “amplification” refers to any in vitro method for increasing the number of copies of a nucleotide sequence with the use of a DNA polymerase. Nucleic acid amplification results in the incorporation of nucleotides into a DNA molecule or primer, thereby forming a new DNA molecule complementary to a DNA template. The formed DNA molecule and its template can be used as templates to synthesize additional DNA molecules. As used herein, one amplification reaction may consist of many rounds of DNA replication. DNA amplification reactions include, for example, polymerase chain reaction (PCR). One PCR reaction may consist of 30-100 “cycles” of denaturation and synthesis of a DNA molecule.

As used herein, “thermostable” refers to a DNA polymerase which is resistant to inactivation by heat. DNA polymerases synthesize the formation of a DNA molecule complementary to a single-stranded DNA template by extending a primer in the 5′-to-3′ direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable DNA polymerase activity is more resistant to heat inactivation than a mesophilic DNA polymerase. However, a thermostable DNA polymerase does not mean to refer to an enzyme which is totally resistant to heat inactivation, and thus heat treatment may reduce the DNA polymerase activity to some extent. A thermostable DNA polymerase typically will also have a higher optimum temperature than mesophilic DNA polymerases.

The present invention is directed to a recombinant DNA molecule having a mutated DNA sequence encoding a protein which has DNA polymerase activity and which incorporates dideoxynucleotides about as well as deoxynucleotides. The mutant DNA molecule of the invention may also contain expression control elements, e.g. a promoter and/or an origin of replication. In this combination, a promoter and the structural gene are positioned and orientated with respect to each other such that the structural gene may be expressed in a host cell under the control of the promoter. The origin of replication is capable of maintaining the promoter/structural gene/origin of replication combination in a host cell. Preferably, the promoter and the origin of replication are functional in the same host cell, such as an E. coli host cell. The DNA molecule is preferably a transformed host cell, exemplified herein by an E. coli host cell (in particular, E. coli DH10B), but may also exist in vitro. The promoter may be any constitutive or inducible promoter. Examples of constitutive promoters that may be used in the practice of the invention include ribosomal protein promoter, RPSL, and the ampicillin resistance gene promoter. Examples of inducible promoters include the lambda P_{L} promoter, tac promoter, and lac promoter. The expressed protein of the invention may have a processive 3′-to-5′ DNA exonuclease activity or may have substantially reduced 3′-to-5′ exonuclease activity. The expressed protein of the invention may also have a 5′-to-3′ DNA exonuclease activity or may have substantially reduced 5′-to-3′ DNA exonuclease activity. The expressed protein of this invention may also have both substantially reduced processive 3′-to-5′ DNA exonuclease activity and substantially reduced 5′-to-3′ DNA exonuclease activity. Preferably, the structural gene is expressed under the control of a heterologous promoter. In addition, the structural gene may be expressed under the control of a heterologous ribosome binding site, although the native DNA polymerase ribosomal binding site may also be used.

The present invention pertains both to the mutant DNA polymerase and to its functional derivatives. The term “functional derivative” is intended to include the “fragments,” “variants,” “analogues,” and “chemical derivatives” of a molecule. A “fragment” of a molecule such as a DNA polymerase, is meant to refer to any polypeptide subset of the molecule. A “variant” of a molecule such as a DNA polymerase is meant to refer to a molecule substantially similar in structure and function to either the entire molecule, or to a fragment thereof A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules is not found in the other, or if the sequence of amino acid residues is not identical. An “analogue” of a molecule such as a DNA polymerase is meant to refer to a molecule substantially similar in function to either the entire molecule or to a fragment thereof As used herein, a molecule is said to be a “chemical derivative” of another molecule when it contains additional chemical moieties not normally a part of the molecule. Such moieties may improve the molecule's solubility, absorption, biological half life, etc.

The present invention also relates to a method for the production of a protein having a mutant DNA polymerase activity as described herein by the steps of culturing a cell containing a mutant DNA molecule of the invention under conditions where the DNA is expressed, followed by purifying the protein expressed during the culturing step. In this method, the recombinant DNA molecule encodes the protein, and also includes a promoter and an origin of replication. (The promoter and the structural gene are in such position and orientation with respect to each other that the promoter may regulate the expression of the gene in the cell). The origin of replication may be heterologous to the structural gene and capable of maintaining the structural gene/promoter/origin of replication combination in the host cell. Preferably, the mutant DNA polymerase gene is expressed and maintained in an E. coli host cell. The promoter may be heterologous to the structural gene and may be inducible, e.g. a lambda P_{L} promoter, a tac promoter, or a lac promoter. Preferably, the structural gene is under control of a heterologous promoter. The structural gene of the invention may be under control of a heterologous ribosome binding site. The protein may have a processive 3′-to-5′ DNA exonuclease activity or may have substantially reduced 3′-to-5′ exonuclease activity. The protein may also have 5′-to-3′ exonuclease activity or may have substantially reduced 5′-to-3′ exonuclease activity. The protein may have both substantially reduced 3′-to-5′ exonuclease activity and substantially reduce 5′-to-3′ exonuclease activity.

Although specific plasmids, vectors, promoters and host cells are disclosed and used in the Example section, other promoters, vectors, and host cells, both prokaryotic and eukaryotic, are well known in the art and in keeping with the specification, may be used to practice the invention. Eukaryotic cells include yeast, CHO, and BHK. Prokaryotic cells include E. coli, Samonella, Baccillus and Streptomyces. Specific molecules exemplified herein include pTTQ-Taq, pSportT5-3, pUC-TneFY, pTrcTne35FY, pTTQTne535FY, pTTQTne5FY, and pTrcTneFY, and functional derivatives thereof A functional derivative of a DNA molecule is derived from the original DNA molecule but still may express the desired mutant DNA polymerase structural gene in a host or in vitro according to the present invention.

The present invention further relates to a mutant DNA polymerases produced by the method of the present invention, having substantially reduced exonuclease activities. Standard protein purification techniques well known in the art may be used to purify the polymerase proteins of the present invention. Preferably, the exonuclease activity is less than about 1 unit/mg protein. More preferably, the exonuclease activity is less than about 0.1 units/mg protein. Even more preferably, the exonuclease activity is less than about 0.003 units/mg protein. Most preferably, the exonuclease activity is less than about 0.0001 units/mg protein.

The amino acid sequences of the DNA polymerases were compared with other known DNA polymerases, such as E coli DNA polymerase I, Taq DNA polymerase, T5 DNA polymerase, and 17 DNA polymerase to localize the regions of 3′-to-5′ exonuclease activity as well as the polymerase and dNTP binding domains. Based on this comparison of the amino acid sequences of various DNA polymerases (Blanco et al., Gene 112:139-144 (1992); Braithwaite and Ito, Nucleic Acids Res. 21:787-802 (1993)), a 3′-to-5′ exonuclease domain was localized as follows:

| | | * | |
|---|---|---|---|
| Tne | 317 | PSFALDLETSS | 327¹ |
| Pol I | 350 | PYFAFDTETDS | 360 |
| T5 | 133 | GPVAFDSETSA | 143 |
| T7 | 1 | -MIVSDIEANA | 10 |

¹Numbering is as reported in U.S.S.N. 08/370,190, filed Jan. 9, 1995.

Mutations, such as insertions, deletions, and substitutions, within this domain can result in substantially reduced 3′-to-5′ exonuclease activity. By way of example, Asp³²² (Tne), Asp³⁵⁵ (PolI), Asp¹³⁸ (T5), and Asp5 (T7), noted by an *, can be converted to Ala³²², Ala²⁵⁵, Ala¹³⁸, and Ala⁵, respectively, to obtain mutants with substantially reduced 3′-to-5′ exonuclease activity.

The mutant DNA polymerase of the invention may have 5′-to-3′ exonuclease activity or may have substantially reduced 5′-to-3′ exonuclease activity. In most of the known polymerases, the 5′-to-3′ exonuclease domain is present at the N-terminal region of this polymerase. Ollis, D L et al., Nature 313:762-766 (1985); Freemont et al., Protein 1:66-73 (1986); Joyce, C. M., Curr. Opin. Struct. Biol. 1:123-129 (1991). There are some conserved amino acids that have been implicated as responsible for 5′-to-3′ exonuclease activity Gutman and Minton, Nucl. Acids Res. 21:44064407 (1993). In E. coli PolI the amino acids include Tyr⁷⁷, Gly¹⁰³, Gly¹⁸⁴ and Gly192. The 5′-to-3′ exonuclease domain is dispensible. The best known example is the Klenow fragment of E coli polymerase I. The Klenow fragment is a natural proteolytic fragment devoid of 5′-to-3′ exonuclease activity. Joyce, C. M. , et al., J. Biol. Chem. 257:1958-64 (1990). For example, the 219 N-terminal amino acid residues of the Tne DNA polymerase can be deleted to result in a mutant with substantially diminished 5′-to-3′ exonuclease activity.

The mutant DNA polymerases of this invention may be used in cloning and in vitro gene expression experiments to produce heterologous polypeptides from the cloned genes. The mutant-DNA polymerases of this invention may also be used for DNA sequencing, DNA labeling, and amplification reactions.

As is well known, sequencing reactions, such as dideoxy DNA sequencing in cycle DNA sequencing of plasmid DNA, require the use of DNA polymerases. Dideoxy-mediated sequencing involves the use of a chain-termination technique which uses a specific polymer for extension of DNA polymerase, a base-specific chain terminator, and the use of polyacrylamide gels to separate the newly synthesized chain-terminated DNA molecules by size so that at least a part of the nucleotide sequence of the original DNA molecule can be determined. Specifically, a DNA molecule is sequenced by using four separate DNA sequencing reactions, each of which contains different base-specific terminators. For example, the first reaction will contain a G-specific terminator, the second reaction will contain a T-specific terminator, the third reaction will contain an A-specific terminator, and a fourth reaction may contain a C-specific terminator. Preferred terminator nucleotides include dideoxyribonucleoside triphosphates (ddNTPs) such as ddATP, ddTTP, ddGTP, and ddCTP. Analogues of dideoxyribonucleoside triphosphates may also be used and are well known in the art.

When sequencing a DNA molecule, ddNTPs lack a hydroxyl residue at the 3′ of the deoxyribonucleoside, and thus, although they can be incorporated by DNA polymerases into the growing DNA chain, the absence of the 3′ hydroxyl residue prevents formation of a phospho-diester bond, resulting in termination of extension of the DNA molecule. Thus, when a small amount of one ddNTP is included in a sequencing reaction mixture, there is a competition between extension of the chain and base-specific termination, resulting in a population of synthesized DNA molecules which are shorter in length than the DNA template to be sequenced. By using four different ddNTPs and four separate enzymatic reactions, populations of the synthesized DNA molecules -can be separated by size so that at least a part of the nucleotide sequence of the original DNA molecule can be determined. DNA sequencing by dideoxy nucleotides is well known, and is described by Sambrook et al. in: Molecular Cloning: A Laboratory Manual, Cold Spring Harbor Laboratory Press, Cold Spring Harbor, N.Y. (1989). As will be readily recognized, the DNA polymerases of the present invention may be used in such sequencing reactions.

As is well known, detectably labeled nucleotides are typically included in sequencing reactions. Any number of labeled nucleotides can be used in sequencing or labeling reactions, including, but not limited to, radioactive isotopes, fluorescent labels, chemiluminescent labels, bioluminescent labels, and enzyme labels. It has been unexpectedly discovered that the Tne DNA polymerase of the present invention may be particularly useful for incorporating αS nucleotides during sequencing or labeling reactions. For example, α³⁵[S]dATP, a commonly-used detectably-labeled nucleotide in sequencing reactions, is incorporated three times more efficiently with the Tne polymerase of the present invention than with Taq DNA polymerase. Thus, the enzymes of the present invention are suited for sequencing or labeling DNA molecules with α³⁵dNTPs. Particularly suited is Tne DNA polymerase or mutants thereof

Polymerase chain reaction (PCR), a well-known DNA amplification technique, is a process by which DNA polymerase and deoxyribonucleoside triphosphates are used to amplify a target DNA template. In such PCR reactions, two primers, one complementary to the 3′ termini (or near the 3′ termini) of the first strand of the DNA molecule to be amplified, and a second primer complementary to the 3′ termini (or near the 3′ termini) of the second strand of the DNA molecule to be amplified, are hybridized to their respective DNA molecules. After hybridization, DNA polymerase, in the presence of deoxyribonucleoside triphosphates, allows the synthesis of a third DNA molecule complementary to the first strand, and a fourth DNA molecule complementary to the second strand of the DNA molecule to be amplified. This synthesis results in two double-stranded DNA molecules. Such double-stranded DNA molecules may then be used as DNA templates for synthesis of additional DNA molecules by providing a DNA polymerase, primers, and deoxyribonucleoside triphosphates. As is well known, the additional synthesis is carried out by “cycling” the original reaction (with excess primers and deoxyribonucleoside triphosphates), allowing multiple denaturing and synthesis steps. Typically, denaturing of double-stranded DNA molecule to form single-stranded DNA templates is accomplished by high temperatures. For example, the Tne DNA polymerase of the present invention is a heat-stable DNA polymerase and thus will survive such thermocycling during DNA amplification reactions. Thus, the Tne DNA polymerase is suited for PCR reactions, particularly where high temperatures are used to denature the DNA molecules during amplification. In addition to Taq, wild type and mutant Thermus flavus, Thermus thermophilus, and Thermus aquaticus DNA polymerases are useful for PCR.

The DNA polymerases of the invention are ideally suited for the preparation of a kit. Kits comprising the DNA polymerase may be used to detectably label DNA molecules, for DNA sequencing, or for DNA amplification by well-known techniques. Such kits may comprise a carrying means being compartmentalized to receive, in close confinement, one or more container means such as vials, test tubes, and the like. Each of such container means comprises components or a mixture of components needed to perform DNA sequencing, DNA labeling, or DNA amplification.

A kit for sequencing DNA may comprise a number of container means. A first container means may, for example, comprise a substantially purified DNA polymerase of the invention. A second container means may comprise one or a number of types of nucleotides needed to synthesize a DNA molecule complementary to a DNA template. A third container means may comprise one or a number of different types of ddNTs. In addition to the above container means, additional container means may be included in the kit comprising one or a number of DNA primers.

A kit used for amplifying DNA will comprise, for example, a first container means comprising a substantially pure DNA polymerase and one or a number of additional container means which comprise a single type of nucleotide or mixtures of nucleotides. Various primers may or may not be included in a kit for amplifying DNA.

When desired, the kit of the present invention may also include container means which comprise detectably labeled nucleotides which may be used during the synthesis or sequencing of a DNA molecule. One or a number of labels may be used to detect such nucleotides. Illustrative labels include, but are not limited to, radioactive isotopes, fluorescent labels, chemiluminescent labels, bioluminescent labels, and enzyme labels.

Having now generally described this invention, the same will be better understood by reference to specific examples, which are included herein for purposes of illustration, and are not intended to be limiting unless otherwise specified.

### EXAMPLES

The overall cloning strategy used in the Examples may be more easily understood by reference to the Figures.

### Example 1

### Preparation of Non-discriminating Mutant DNA Polymerases

As models, T5, Tne, and Taq DNA polymerases were used. The polymerase active site, including the DNTP binding domain, is usually present in the C-terminal region of the polymerase (Ollis, D. L., et al., Nature 313:763-766 (1985); Freemont, P. S., et all., Proteins 1: 66-73 (1986).) Our partial sequence of the Tne polymerase gene suggests that the amino acids that presumably contact and interact with the dNTPs are present within the 694 bases starting of the internal BamHI site, based on the homology with the prototype polymerase E. Coli PolI (Poleskey A. H., et al., J. Biol. Chem. 265:14579-14591 (1990). The corresponding amino acids in other polymerases are present in the O helix.

Initially, it was attempted to replace amino acids 544 to 729 (coordinates from Leavitt and Ito, Proc. Natl. Acad. Sci USA 86:44654469 (1989)) of T5 DNA polymerase with amino acids 500 to 675 (coordinates from Dunn and Studier, J. Mol. Biol. 166: 477-535 (1983)) of T7 DNA polymerase. This region encompasses entire O-helix plus additional amino acids on either side of the helix. The extra amino acids were chosen for convenient restriction sites DraIII and SspI present in T5 DNA polymerase. The corresponding region of T7 was generated by PCR using the oligos:

[SEQ. ID. No. 1]:
5′-CAGGATCCACATGGTGCTTAACGGCGACATCCACACTAAG
and
[SEQ. ID. No. 2]:
GTTAACTTCTTGTGCGGTCTCAATGAC.

The hybrid plasmid containing the active sites of T7 DNA polymerase was constructed by replacing the T5 active sites with the PCR product. However, the construct did not produce any active protein, perhaps because the structure of the altered protein was unstable in E. coli. Therefore, it was reasoned that it may be possible to change specific amino acids of T5 DNA polymerase and Taq DNA polymerase in the O-helix (based on the sequence comparison with the T7 DNA polymerase) to produce an active hybrid polymerase. This small change should not alter significantly the structure of the mutant polymerase.

The amino acid sequence in the O-helix of T7, T5, Tne, Taq, and the Klenow fragment are as follows:

| | | | | |
|---|---|---|---|---|
| Tma | 725 | GKMVNFSIIYG | 735 | [SEQ ID No. 17] |
| T5 | 565 | AKAITFGILYG | 675 | [SEQ ID No. 3] |
| T7 | 521 | AKTFIYGFLYG | 531 | [SEQ ID No. 4] |
| Taq | 662 | AKTINFGVLYG | 672 | [SEQ ID No. 5] |
| Klenow frag. | 757 | AKAINFGLIYG | 767 | [SEQ ID No. 6] |
| Tne | 62 | GKMVNFSIIYG | 72 | [SEQ ID No. 12] |

The sequence of the Klenow fragment is disclosed by Polesky, A. H. et al., J. Biol. Chem. 265:14579-14591 (1990), and the sequence of the C-terminal portion of the Tne polymerase gene is shown in FIG. 4.

T7 DNA polymerase has a sequence stretch Thr-Phe-Ile-Tyr [SEQ ID No. 7] in the O-helix. The corresponding sequence in T5, Taq, and Tne DNA polymerase are Ala-Ile-Thr-Phe [SEQ ID No. 8]; Thr-Ile-Asn-Phe [SEQ ID No. 9]; and Met-Val-Asn-Phe [SEQ ID No. 13], respectively. These amino acid are bordered by known conserved dNTP binding amino acids Lys (K) and Tyr (Y). Therefore, it was tested whether changing these amino acids of T5 and Taq DNA polymerases to Thr-Phe-Ile-Tyr [SEQ ID No. 7] would make the polymerases as non-discriminating as T7 DNA polymerase. One of the main differences in this region of T7 DNA polymerase is that it contains a tyrosine residue with an hydroxyl group in place of phenylalanine in the case of Klenow fragment, T5 and Taq DNA polymerases. An oligo T CAG GCT GCT AAA ACA TTC ATC TAC GGT ATA CTG TAT GGT TCT GG [SEQ ID No. 10] was generated to change Ala-Ile-Thr-Phe [SEQ ID No. 8] of T5 DNA polymerase to Thr-Phe-Ile-Tyr [SEQ ID No. 7] by site directed mutagenesis. The oligo was also designed to create an AccI site to detect the mutant clone in the process. The mutagenesis was done using BioRad Mutagene Kit (BioRad, California) according to the protocol described by the manufacturer.

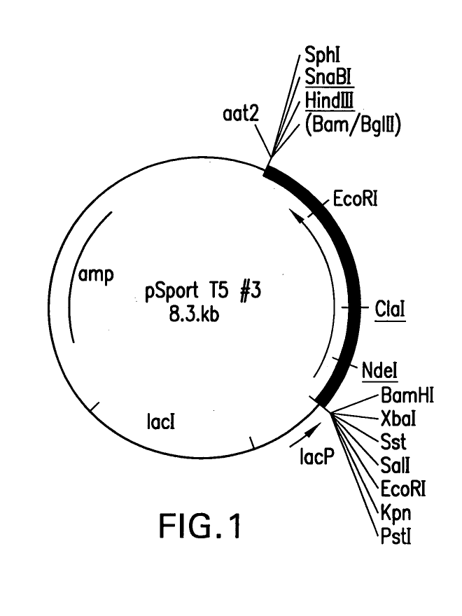

Protocol for mutagenesis: pSport T5-E (FIG. 1) was digested with ClaI and EcoRI to generate a 1.9 kb fragment of T5 DNA polymerase. The fragment was cloned onto M13mp18 (LTI, Gaithersburg, Md.) at the AccI and EcoRI sites. The recombinant clone was selected in DH5αF′IQ (LTI, Gaithersburg, Md.). Single stranded uracilated DNA was isolated from CJ236 (Biorad, Calif.) and used for site-directed mutagenesis using the Biorad Mutagene kit. Following mutagenesis, 6 clones were tested for the presence of an additional AccI site included in the mutagenic oligo [SEQ ID No. 7]. Five of the six clones produced about a 1 kb fragment, an indication that these clones contain expected mutations. One of the clones was used to replace the wild type fragment in pSport T5-E. First, a 1.0 kb DraIII-EcoRI fragment of wild type T5 DNA polymerase of pUC#3-Exo (which contains the BamHI-EcoRI fragment of T5 DNA polymerase gene from pSportT5-E in pUC19) replaced with the DraIII-EcoRI fragment of the mutant M13 RF DNA. This fragment contains the mutations. Second, EcoRI-6 fragment T5 Phage DNA which contains the residual COOH-end of the T5 polymerase gene (U.S. Pat. Nos. 5,270,179 and 5,047,342) was cloned in pUCT5 mutant in order to reconstruct the entire T5 DNA polymerase gene. Finally, the entire T5 polymerase gene containing the mutations (AITF to TFIY) was cloned in pSport (LTI, Gaithersburg, Md.) at the BamHI site as BamHI-BglII as described before (U.S. Pat. Nos. 5,270,170 and 5,047,342). An active hybrid (T5/T7) polymerase was obtained from the recombinant clone.

Similarly, an oligo GTA GAG GAC CCC GTA ATT AAT GGT CTT GGC CGC [SEQ ID No. 11] was designed to change the phenylalanine residue (amino acid 667) to a tyrosine of Taq DNA polymerase. An AseI site was also created for initial screening of the mutant clones.

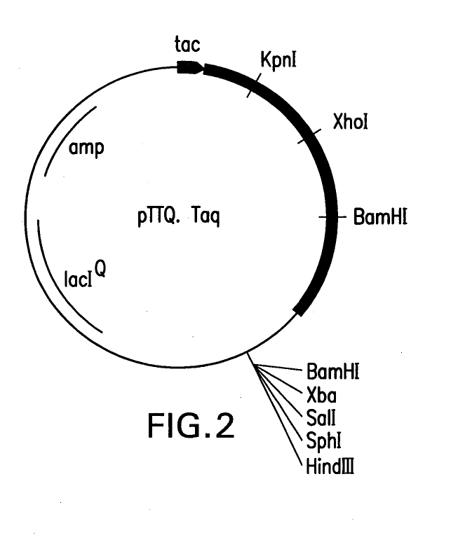

Since thermostable Taq DNA polymerase cloned and expressed in E. coli can be purified very easily, the mutant Taq DNA polymerase was characterized with respect to its DNA polymerase activity and its ability to produce sequencing ladder in the presence of varying amount of dideoxynucleotides. A 2.5 kb portion of Taq DNA polymerase (FIG. 2) was cloned as a HindIII-XbaI fragment in M13mp19 (LTI, Gaithersburg, Md.). The kinased oligo was used for mutagenesis by the procedure as described above. Following mutagenesis the mutant fragment was cloned in the expression vector as follows.

The DNA fragment from the mutant phage DNA was obtained by digesting the DNA with NgoAIV and XbaI. The 1.6 kb NgoAIV-XbaI fragment of pTTQ-Taq was replaced with the 1.6 kb NgoAIV-XbaI fragment containing the mutation (F667Y). The mutant clone produced active polymerase.

Upon testing in the DNA sequencing reaction optimized for wild type Taq DNA polymerase, it was found that the mutant Taq DNA polymerase is unable to produce satisfactory ladder and the DNA sequencing (synthesis) is terminated prematurely. With the mutant Taq DNA polymerase, only nine bases of pUC18 DNA sequence were able to be read. The wild type Taq DNA polymerase produced expected sequencing ladder under identical conditions (we were able to read up to 300-4400 bases of pUC18 DNA sequence). This is an indication that the mutant polymerase is incorporating dideoxynucleotides very efficiently and the DNA sequencing reaction is terminating prematurely. By decreasing the concentration of dideoxynucloeside triphosphates, it was possible to generate a sequencing ladder. (When dNTPs concentrations were held constant to 20 μM, and the ddNTP concentrations were reduced 100-fold, >400 bases of sequence in the G-lane (0.4 μM) were read. The other ddNTPs needed to be reduced even further as their initial concentrations were 5-10 fold higher.) The mutant Taq DNA polymerase needed 100-fold less dideoxynucleotides compared to the wild type Taq DNA polymerase to generate DNA sequencing ladder. This suggests that the mutant Taq DNA polymerase became nondiscriminatory upon modification of phenylalanine 667 to tyrosine 667. In addition, the mutant Taq DNA polymerase produced almost uniform band intensity compared to the wild-type in the sequencing ladder in the presence of chain terminating dideoxynucleotides. The result suggests that uneven band intensity in the sequencing ladder was at least in part due to discriminatory activity towards nonnatural nucleotides.

An attempt was made to generate a similar mutant of Tne DNA polymerase. It was anticipated that the mutant Tne DNA polymerase will be better in DNA sequencing reactions because Tne DNA polymerase inherently incorporates [∝S³⁵] dNTs 3-to-5 fold better than the Taq polymerase, a property highly desirable in DNA sequencing. In order to change the Phe⁶⁷to a Tyr⁶⁷ (FIG. 4; SEQ ID No. 14) site-directed mutagenesis was performed using the oligonucleotide.
GTA TAT TAT AGA GTA GTT AAC CAT CTT TCCA. [SEQ ID No. 15]

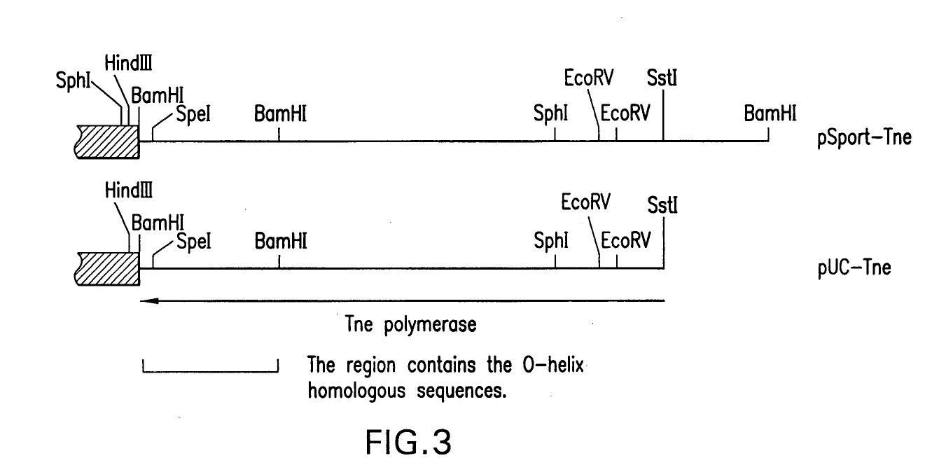

In this oligonucleotide a HpaI restriction site was introduced to facilitate screening of the mutants. To make a mutant Tne DNA polymerase, a 2 kb SphI fragment of pSport -Tne FIG. 3) was cloned into M13mp19 (LTI, Gaithersburg, Md.). The recombinant was selected in E. coli DH5aF′IQ (LTI, Gaithersburg, Md.). One of the clones with a proper insert was used to isolate uracilated single-stranded DNA by infecting E. coli CJ236 (Biorad, Calif.), with a phage particle obtained from E. coli DH5aF′IQ. A single-stranded uracilated DNA was used for site-directed mutagenesis using the protocol described in the BioRad manual, see supra, except T7 DNA polymerase was used instead of T4 DNA polymerase. The resulting mutants were screened for the presence of the HpaI site. Mutants with the desired HpaI site were used for further study.

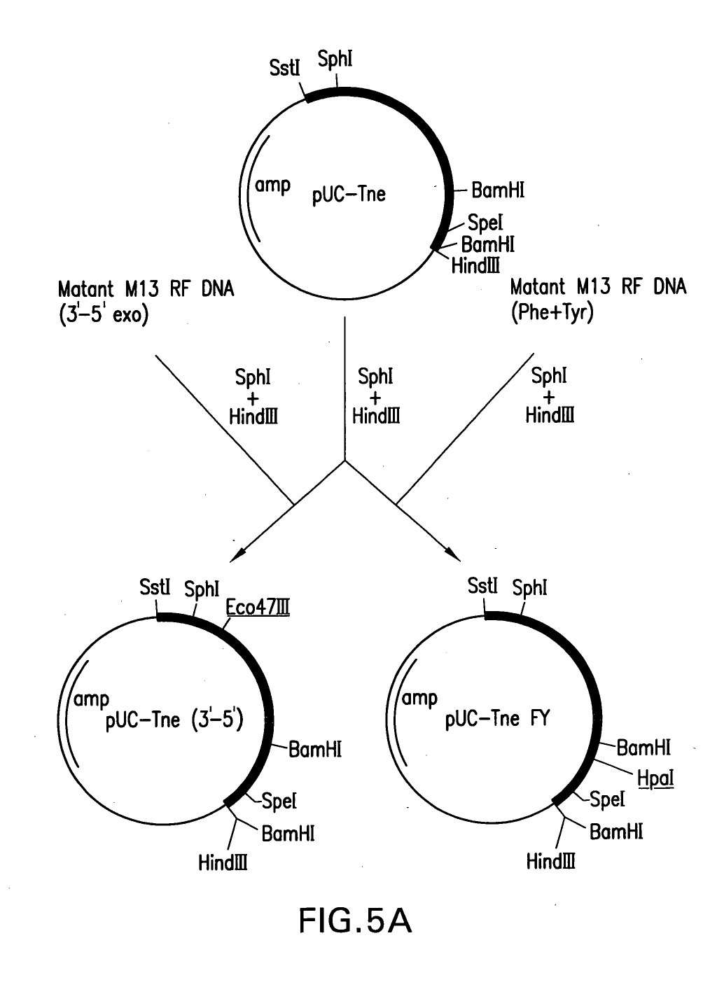

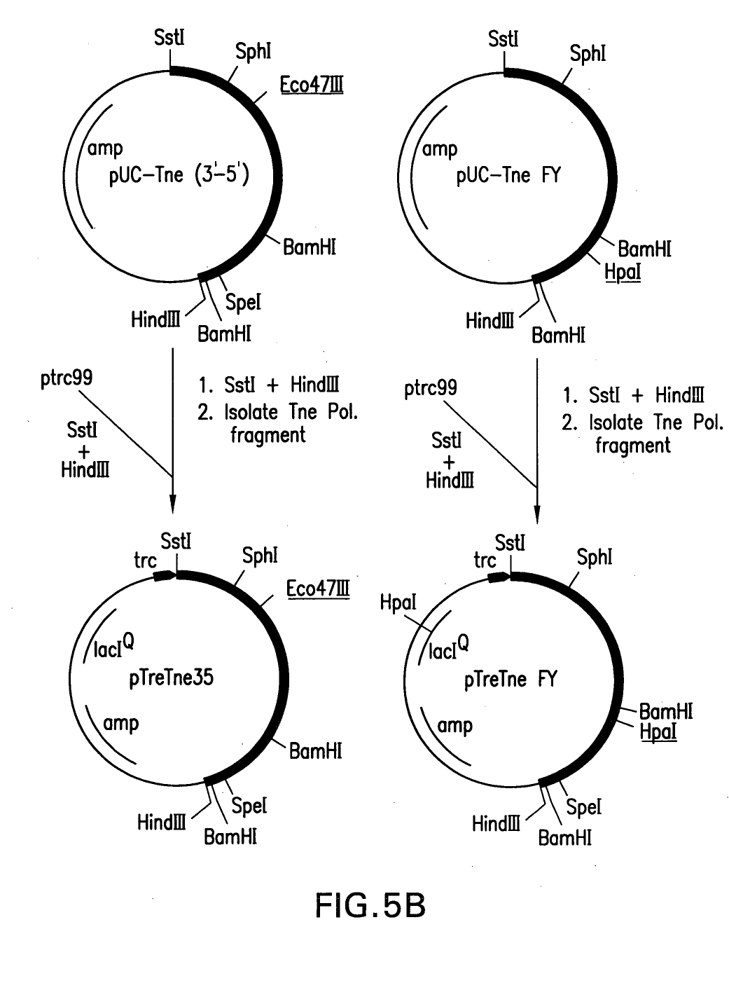

DNA containing the Phe⁶⁷→Tyr⁶⁷ mutations were incorporated into pUC-Tne by replacing the wild type SphI-HindIII fragment with the mutant fragment obtained from the mutant phage DNA from the site-directed mutagenesis. The structure of the desired clone, pUC-Tne FY, was confirmed by the presence of the unique HpaI site. (FIG. 5A) The entire mutant polymerase gene was subcloned into pTrc99. The plasmid, pUC-TneFY, was digested with SstI and HindIII and the entire mutant polymerase gene (2.6 kb) was purified and cloned with SstI and HindIII digested pTrc99 expression vector (Pharmacia, Sweden). The clones were selected in DHH10B (LTI, Gaithersburg, Md.). The desired plasmid was designated pTrcTneFY (FIG. 5B). The clone produced active heat stable polymerase.

The purification of the mutant Tne polymerase was done essentially as described in U.S. patent application, Ser. No. 08/370,190, filed Jan. 9, 1995, incorporated by reference herein, with minor modifications. Five to 10 grams of cells expressing the cloned mutant Tne DNA polymerase were lysed by sonication with a Heat Systems Ultrasonic Inc. Model 375 sonicator in a sonication buffer consisting of 50 mM Tris-HCl, pH 7.4; 8% glycerol, 5 mM 2-mercaptoethanol, 10mnM NaCl, 1 mM EDTA, 0.5 mM PMSF. The sonicated sample was heated at 75° C. for 15 min. Following heat treatment, 200 MM NaCl and 0.4% PEI was added to remove nucleic acids. The extract was centrifuged for clarification. Ammonium sulfate was added to 48%, the pellet was resuspended in a column buffer consisting of 25 mM Tris-HCl, pH 7.4, 8% glycerol, 0.5% EDTA, 5 mM 2-mercaptoethanol, 10 mM KCl, and loaded on a Heparin column. The column was washed with 10 column volumes of a buffer gradient from 10 mnM to 1 M KCl. Fractions containing polymerase activity were pooled and dialyzed in column buffer as above except the pH is 7.8. The dialyzed pooled fractions were loaded onto a MonoQ column. The column was washed and eluted as described above. The active fractions are pooled and a unit assay was done. The reaction contained 25 mM TAPS, pH 9.3, 2 mM MgCl₂, 50 mM KCl, 1 mM DTT, 0.2 mM dNTPs, 500 μg/ml DNase I treated salmon sperm DNA, 21 mCi/ml [αP³²] dCTP and various amount of polymerase in a final volume of 50 ml. After 10 min. at 70° C., 10 ml of 0.5 M EDTA was added to the tube. TCA precipitable counts were measured in GF/C filters using 40 ml of the reaction.

Upon testing in the DNA sequencing reaction, the TneFY mutant polymerase gave only a 9 base sequencing ladder when the Taq cycle sequencing reaction conditions were used (LTI). Diluting the dideoxynucleotides by a factor of 100 extended the ladder to about 200 bases. The F→Y mutation in the TneFY polymerase, therefore, allowed dideoxynucleotides to be incorporated at a much higher frequency than for wild-type polymerase. Taken together, it can be concluded that T5, Taq, Tne, Tma and other DNA polymerases can be made nondiscriminatory towards dideoxynucleotide and perhaps other nonnatural nucleotides by simple modification of a specific phenylalanine residue to a tyrosine residue. These DNA polymerases are useful in DNA sequencing and other molecular biological applications.

### Example 2

### Preparation of Non-discriminating Mutant DNA Polymerase Substantially Reduced in 3′ to-5′ Exonuclease Activity

To make the 3′-to-5′ exonuclease mutants, an oligonucleotide, GA CGT TTC AAG CGC TAG GGC AAA AGA [SEQ ID No. 16] was used to convert the Asp³²² to Ala³²². An Eco47III site was created to facilitate screening of the mutant following mutagenesis. The mutagenesis was performed using a protocol as described in the Biorad manual except T7 DNA polymerase was used instead of T4 DNA polymerase. See supra. The mutant clones were screened for an Eco47III site that was created in the mutagenic oligonucleotide. One of the mutants having the created Eco47III site was used for further study.

To incorporate the 3′-to-5′ exonuclease mutation into an expression vector, the mutant phage DNA obtained as described above was digested with SphI and HindIII and a 2 kb fragment containing the mutation was isolated. The fragment was cloned in pUC-Tne to replace the wild-type fragment (FIG. 5A). The desired clone, pUC-Tne (3′→5′) was confirmed by the presence of a unique Eco47III site. The plasmid digested with SstI and HindIII in the entire mutant polymerase gene (2.6 kb) was purified and cloned into SstI and HindIII digested pTrc99 expression vector, obtainable from Pharmacia, Sweden. The clones were selected in DH10B (LTI, Gaithersburg, Md.). The desired plasmid was designated as pTrcTne35 (FIG. 5B). The clone produced active heat stable polymerase. The polymerase was purified as described supra, for TneFY in Example 1.

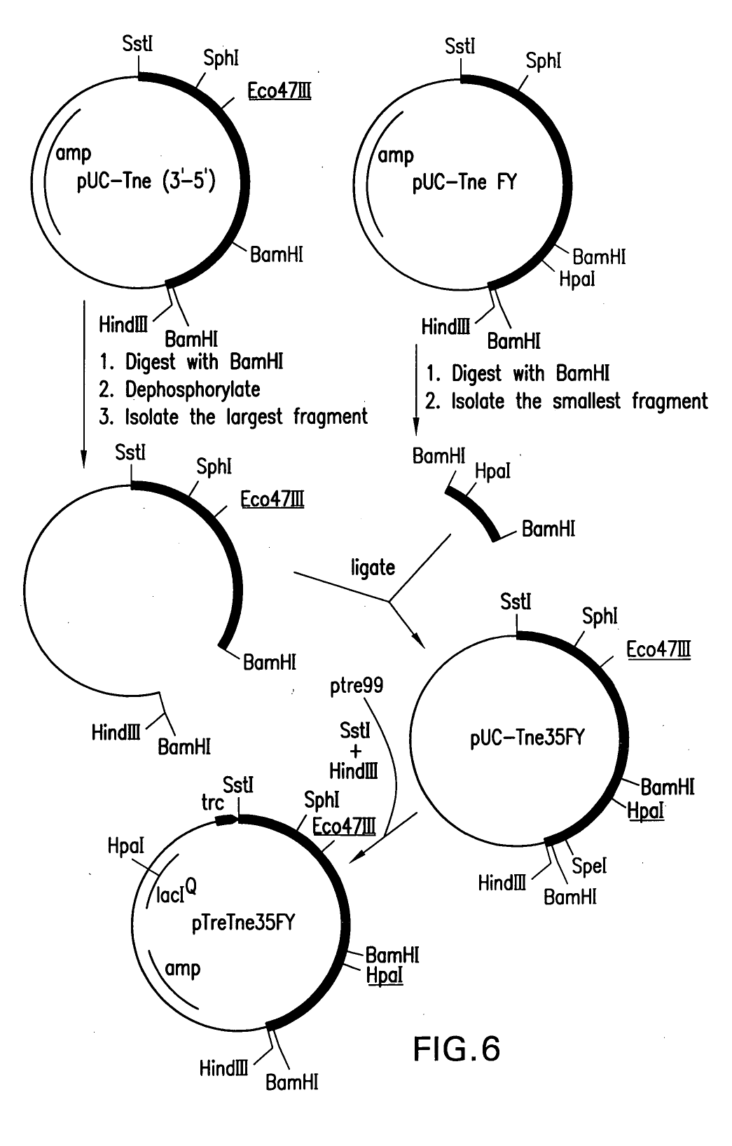

In order to introduce both the 3′-to-5′ exonuclease mutation and the Phe⁶⁷→Tyr⁶⁷ mutation in the expression vector pTrc99, it was first necessary to reconstitute both mutations in a pUC-Tne clone (FIG. 6). Both pUC-Tne (3′-to-5′) and pUC-TneFY were digested with BamHI. The digested pUC-Tne (3′→5′) was desphosphorylated to avoid recircularization in the following ligation step. Both digested plasmids were run in a 1% agarose gel. The largest BamHI fragment (4.4 kb) was purified from pUC-Tne (3′-5′) digested DNA and the small BamHI fragment (0.8 kb) containing the Phe⁶⁷→Tyr⁶⁷ mutation was purified and ligated to generate pUC-Tne35FY. The proper orientation and the presence of both mutations were confirmed by Eco47III HpaI and SphI-HindIII restriction digest (FIG. 6). Finally, the entire polymerase gene containing both mutations was subcloned as an SstI-HindIII fragment in pTrc99 to generate pTrcTne35FY in DH10B. The clone produced active heat stable polymerase. The polymerase was purified as described supra to Example 1.

The Tne3 SFY mutant was used in cycle sequencing reactions using p³² end-labeled primers. This mutant produced a sequencing ladder and exhibited a similar ability to incorporate dideoxynucleotides as TneFY. In this case the sequence extended to beyond 400 bases and the excess p³² end-labeled M13/pUC Forward 23-base Sequencing Primer band remained as a 23-base position in the ladder. The persistence of the 23-base primer band confirmed that the 3′-to-5′ exonuclease activity had been significantly reduced.

### Example 3

### Preparation of Non-discriminating Mutant DNA Polymerases Exhibiting Substantially Reduced 5′ to-3′ Exonuclease Activity

In order to generate an equivalent mutant devoid of 5′-to-3′ exonuclease activity as well as 3′-to-5′ exonuclease activity, the presence of a unique SphI site present 680 bases from the SstI site was exploited. pUC-Tne35FY was digested with HindIII, filled-in with Klenow fragment to generate a blunt-end, and digested with SphI. The 1.9 kb fragment was cloned into an expression vector pTTQ19 at the SphI-SmaI sites and was introduced into E. coli DH10B. (Stark, M. J. R., Gene 51:255-267 (1987)). This cloning strategy generated an in-frame polymerase clone with an initiation codon for methionine from the vector. The resulting clone is devoid of 219 amino terminal amino acids of Tne DNA polymerase. This clone is designated as pTTQTne535FY. The clone produced active heat stable polymerase. No exonuclease activity could be detected in the mutant polymerase as evidence by lack primer degradation previously labeled with radioisotope in the sequencing reaction. The mutant polymerase was purified as described supra, in Example 1. This particular mutant polymerase is highly suitable for DNA sequencing.

Cycle sequencing reactions using p³² end -labeled primers were prepared using this mutant. The sequencing reaction produced sequencing ladders. The Tne535FY mutant performed similarly to the Tne35FY mutant except that the signal intensity increased by at least 5 fold. The background was very low in the relative band intensities were extremely even, showing no patterns of sequence-dependent intensity variation.

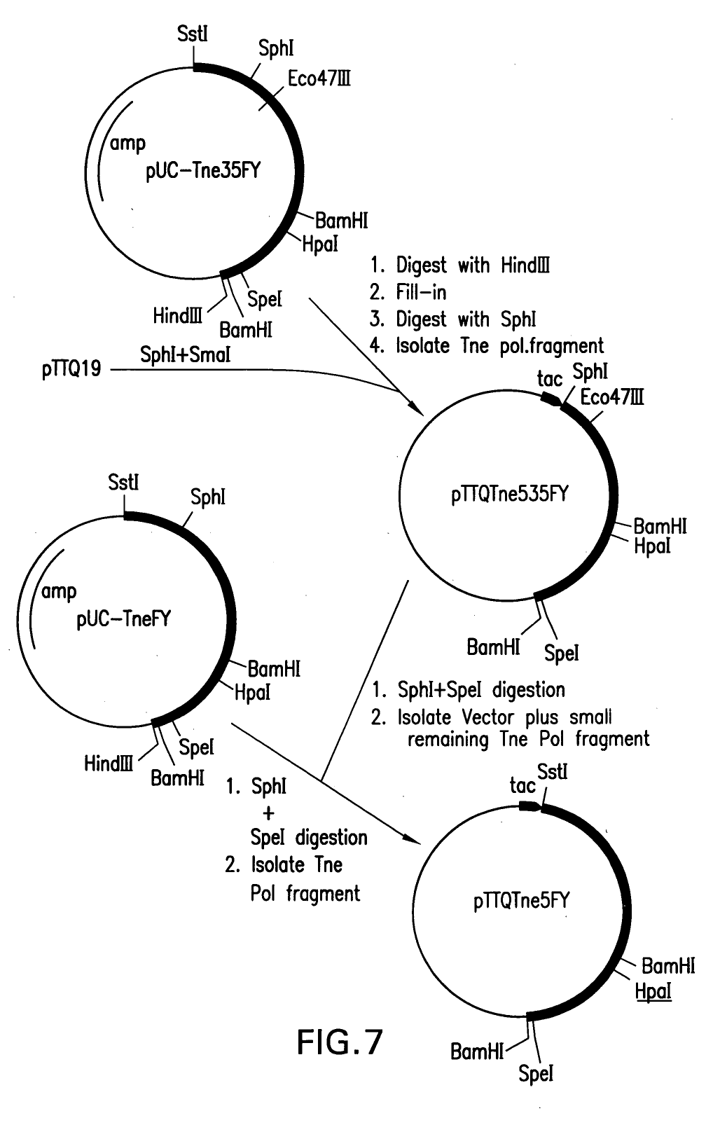

A 5′-to-3′ exonuclease deletion mutant of Tne DNA polymerase containing a Phe⁶⁷→Tyr⁶⁷ mutation was also obtained. In order to generate this mutant, the 1.8 kb SphI-SpeI fragment (FIG. 7) of pTTQTne35FY was replaced with the identical fragment of pUC-TneFY. The clone, pTTQTne5FY, produced active heat stable polymerase. The mutant had modulated, low but detectable, 3′-to-5′ exonuclease activity compared to wild-type Tne DNA polymerase as measured by the rate of degradation of the labeled primer. M13 sequencing primer (LTI, Gaithersburg, Md.) was labeled at the 5′-end with [γ³²] ATP and T4 Kinase (LTI, Gaithersburg, Md.) as described by the manufacturer. The reaction contained 2.0 units of either wild-type or the mutant Tne DNA polymerase, 0.25 pmol of labeled primer, 20 mM Tricine, pH 8.7, 85 mM potassium acetate, 1.2 mM magnesium acetate, and 8% glycerol. Incubation was carried out at 70° C. At various time points, 10 μl aloquots were removed to 5 μl cyclesequencing stop solution and resolved in 6% polyacryamide sequencing gel followed by autoradiography. While the wild-type polymerase degraded the primer in 5 to 15 minutes, it took the mutant polymerase more than 60 minutes for the same amount of degradation of the primer. Preliminary results suggest that this particular mutant polymerase is able to amplify more than 12 kb of genomic DNA when used in conjunction with Taq DNA polymerase. Thus, this mutant polymerase will be suitable for large fragment PCR.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following Claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

1. A DNA molecule comprising a coding sequence for a mutant protein, wherein said mutant protein is a mutant DNA polymerase selected from the group consisting of: E. coli DNA polymerase I, Klenow fragment of E. coli DNA polymerase I, Streptococcus pneumoniae polymerase, Thermus aquaticus polymerase, Thermus flavus polymerase, Thermus thermophilus polymerase, Deinococcus radiodurans polymerase, Bacillus caldotenax polymerase, E. coli bacteriophage T5 polymerase, mycobacteriophage L5 polymerase, Thermatoga maritima polymerase, and E. coli bacteriophage SP01 polymerase, and
wherein said mutant DNA polymerase comprises a substitution of Tyr for Phe at a position in said polymerase corresponding to Phe₅₇₀ of wild-type T5 polymerase.

2. The DNA molecule of claim 1, further comprising a promoter, wherein said promoter is in a position and orientation with respect to the coding sequence such that the mutant protein may be expressed in a cell under the control of said promoter.

3. The molecule of claim 2, wherein said coding sequence is heterologous to said promoter.

4. A host cell comprising the DNA molecule of claim 1.

5. The host cell of claim 4, wherein said host cell is E. coli.

6. A method for producing a protein, wherein said protein is a mutant DNA polymerase selected from the group consisting of: E. coli DNA polymerase I, Klenow fragment of E. coli DNA poymerase I, Streptococcus pneumoniae polymerase, Thermus aquaticus polymerase, Thermus flavus polymerase, Thermus thermophilus polymerase, Deinococcus radiodurans polymerase, Bacillus caldotentex polymerase, E coli bacteriophage T5 polymerase, mycobacteriophage L5 polymerase, Thermatoga maritima polymerase, and E. coli bacteriophage SP01 polymerase, comprising a substitution of Tyr for Phe at a position in said polymerase corresponding to Phe₅₇₀ of wild-type T5 polymerase, said method comprising:
(a) culturing a host cell comprising the DNA molecule of claim 2, and
(b) isolating said protein from said host cell.

7. A mutant DNA polymerase selected from the group consisting of a mutant of: E. coli DNA polymerase I, Klenow fragment of E. coli DNA polymerase I, Streptococcus pneumoniae polymerase, Thennus aquaticus polymerase, Thermus flavus polymerase, Thermus thermophilus polymerase, Deinococcus radiodurans polymerase, Bacillus caldotenax polymerase, E. coli bacteriophage T5 polymerase, Thermatoga maritima polymerase, mycobacteriophage L5 polymerase, and E. coli bacteriophage SP01 polymerase,
wherein said mutant DNA polymerase comprises a substitution of Tyr for Phe at a position in said polymerase corresponding to Phe₅₇₀ of wild-type T5 polymerase.

8. A DNA molecule as claimed in claim 1, wherein said mutant protein is a mutant T5 DNA polymerase comprising a substitution of Tyr for Phe₅₇₀ of wild-type T5 polymerase.

9. The DNA molecule of claim 8, further comprising a promoter, wherein said promoter is in a position and orientation with respect to the coding sequence such that the mutant protein may be expressed in a cell under the control of said promoter.

10. The molecule of claim 8, wherein said coding sequence is heterologous to the promoter.

11. A host cell comprising the DNA molecule of claim 8.

12. The host cell of claim 11, wherein said host cell is E. coli.

13. A method for producing a protein, wherein said protein is a mutant T5 DNA polymerase comprising a substitution of Tyr for Phe₅₇₀ of wild-type T5 polymerase, said method comprising:
(a) culturing a host cell comprising the DNA molecule of claim 9, and
(b) isolating said protein from said host cell.

14. A mutant DNA polymerase as claimed in claim 7, wherein said mutant DNA polymerase is a mutant T5 DNA polymerase comprising a substitution of Tyr for Phe₅₇₀ of wild-type T5 DNA polymerase.

15. A DNA molecule as claimed in claim 1, wherein said mutant protein is a mutant Taq DNA polymerase comprising a substitution of Tyr for Phe₆₆₇ of wild-type Taq polymerase.

16. The DNA molecule of claim 15, further comprising a promoter, wherein said promoter is in a position and orientation with respect to the coding sequence such that the mutant protein may be expressed in a cell under the control of said promoter.

17. The molecule of claim 16, wherein said coding sequence is heterologous to the promoter.

18. A host cell comprising the DNA molecule of claim 15.

19. The host cell of claim 18, wherein said host cell is E. coli.

20. A method for producing a protein, wherein said protein is a mutant Taq DNA polymerase comprising a substitution of Tyr for Phe₆₆₇ of wild-type Taq polymerase, said method comprising:
(a) culturing a host cell comprising the DNA molecule of claim 16, and
(b) isolating said protein from said host cell.

21. A mutant DNA polymerase as claimed in claim 7, wherein said mutant DNA poymerase is a mutant Taq DNA polymerase comprising a substitution of Tyr for Phe₆₆₇ of wild-type Taq DNA polymerase.

22. A DNA molecule as claimed in claim 1, wherein said mutant protein is a mutant Klenow fragment of E. coli DNA polymerase I comprising a substitution of Tyr for Phe₇₆₂ of wild-type Klenow fragment DNA polymerase.

23. The DNA molecule of claim 22, further comprising a promoter, wherein said promoter is in a position and orientation with respect to the coding sequence such that the mutant protein may be expressed in a cell under the control of said promoter.

24. The molecule of claim 23, wherein said coding sequence is heterologous to the promoter.

25. A host cell comprising the DNA molecule of claim 22.

26. The host cell of claim 25, wherein said host cell is E. coli.

27. A method for producing a protein, wherein said protein is a mutant Klenow fragment of E. coli DNA polymerase I comprising a substitution of Tyr for Phe₇₆₂ of wild-type Klenow fragment of E. coli DNA polymerase L said method comprising:
(a) culturing a host cell comprising the DNA molecule of claim 23, and
(b) isolating said protein from said host cell.

28. A mutant DNA polymerase as claimed in claim 7, wherein said mutant DNA polymerase is a mutant Klenow fragment of E. coli DNA polymerase I comprising a substitution of Tyr for Phe₇₆₂ of wild-type Klenow fragment of E. coli DNA polymerase I.
